(19) United States
(12) Patent Application Publication
(10) Pub. No.: US 2011/0168578 A1
(43) Pub. Date: Jul. 14, 2011

(54) BLISTER PACKAGING

(75) Inventor: Gideon Brunner, Basel (CH)

(73) Assignee: STRAUMANN HOLDING AG, Basel (CH)

(21) Appl. No.: 13/060,116

(22) PCT Filed: Aug. 5, 2009
(86) PCT No.: PCT/EP2009/060159

§ 371 Date: Feb. 22, 2011

(30) Foreign Application Priority Data
Aug. 21, 2008 (DE) 20 2008 011 156.0

(51) Int. Cl. A61B 19/02

(52) U.S. Cl. 206/63.5

(57) ABSTRACT

A blister packaging, in particular for dental implants, comprising a web structure to which a rear wall can be fastened in sealing fashion and a plastic film molded part that is made in a single piece together with the web structure, wherein the web structure is equipped with at least two support wings that are angled at about 90° with respect to the web structure and that extend along opposite sides of the web structure.

The present invention relates to a blister packaging and in particular to a blister packaging for dental implants or the like.

### STATE OF THE ART

In the state of the art, blister packaging (display packaging) is known which allows the user of the packaged product to see it. The product is presented in front of a rear wall and fixed into place by means of a transparent plastic film molded part connected to the rear wall which substantially assumes the shape of the product.

In blister packaging, the rear wall can be made of plastics, cardboard or aluminum foil.

The types of blister packaging are grouped into packaging by means of welding, packaging by means of clamping, packaging by means of tacking and packaging by means of adhesive, depending on how they are made. In packaging by means of welding, the plastic film molded part and the rear wall are interconnected by applying heat which at the same time provides a seal for the product. In packaging by means of clamping, the plastic film molded part is circumferentially bent around a rear wall by applying heat. In packaging by means of tacking, the plastic film molded part and the rear wall are interconnected by means of tack rivets. In packaging by means of adhesive, the flat portions of the rear wall and the plastic film molded part are adhesively bonded together.

A widely spread use of blister packaging is in the medical field. The placement of medical products in individual indentations or cavities of the plastic film molded part, sealed tight by the rear wall, has several advantages over glass or plastic vials. It is more hygienic, avoids undesirable influences such as high air humidity or dirt and makes it possible to easily recognize the medical product.

Moreover, special known types of blister packaging allow a display of product data, such as type of application, batch number, manufacturer etc. on the packaging.

With the conventional blister packaging mentioned above, however, there is a problem with vertical stacking, where this problem occurs even more often if the plastic film molded part has an irregular shape.

Also, the indication of product data in an area on the rear wall is limited, even more so if a plurality of blister packages are stacked vertically so that the rear wall is no longer visible in the stack.

### SUMMARY OF THE INVENTION

It is therefore the aim of the present invention to provide a blister packaging, in particular a blister packaging for dental implants or the like, which avoids the above problems.

Within this aim, it is an object of the present invention to provide a blister packaging, in particular a blister packaging for dental implants or the like, which can be easily stacked vertically, at the same time providing a plurality of surfaces for displaying information which concerns the packaged product.

Moreover, another object of the present invention is to provide a blister packaging, in particular a blister packaging for dental implants or the like, having improved stability of shape.

This aim and these and other objects, to be found in the following description, are achieved by a blister packaging, in particular a blister packaging for dental implants or the like, according to the appended claims.

Further characteristics and advantages of the present invention as well as the functioning of the exemplary embodiment of the present invention are described hereafter with reference to the accompanying drawings. The drawings illustrate the present invention and are further intended, together with the description, to explain the basic features of the invention and enable a person skilled in the relevant art to manufacture and use the invention, wherein:

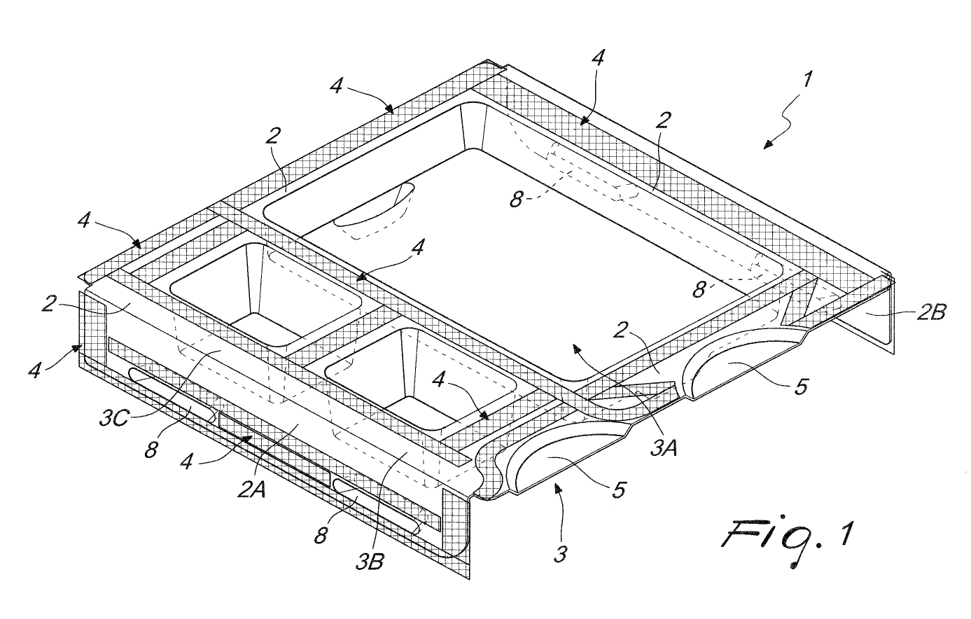

FIG. 1 shows a perspective view of a first embodiment of the blister packaging according to the invention;

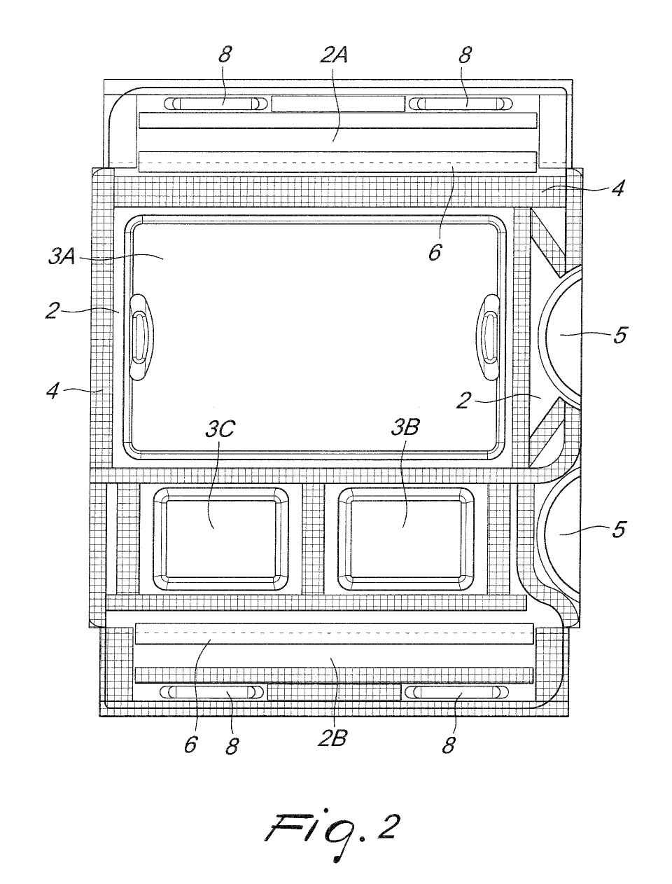

FIG. 2 shows a view of the blister packaging of FIG. 1 from below in a prolate state wherein the support wings are not folded;

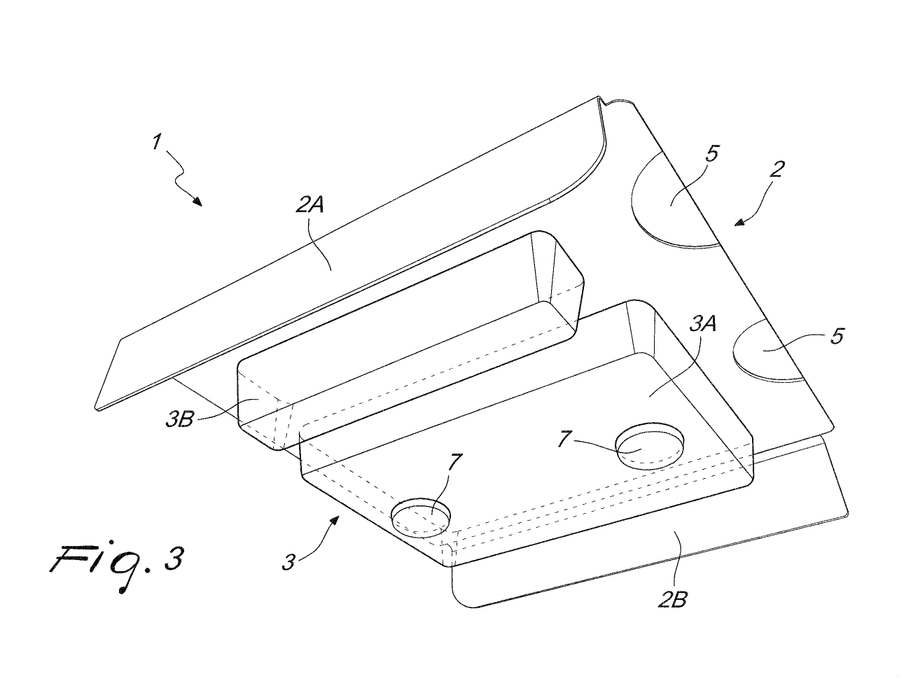

FIG. 3 shows a perspective view of a second embodiment of the blister packaging according to the invention; and

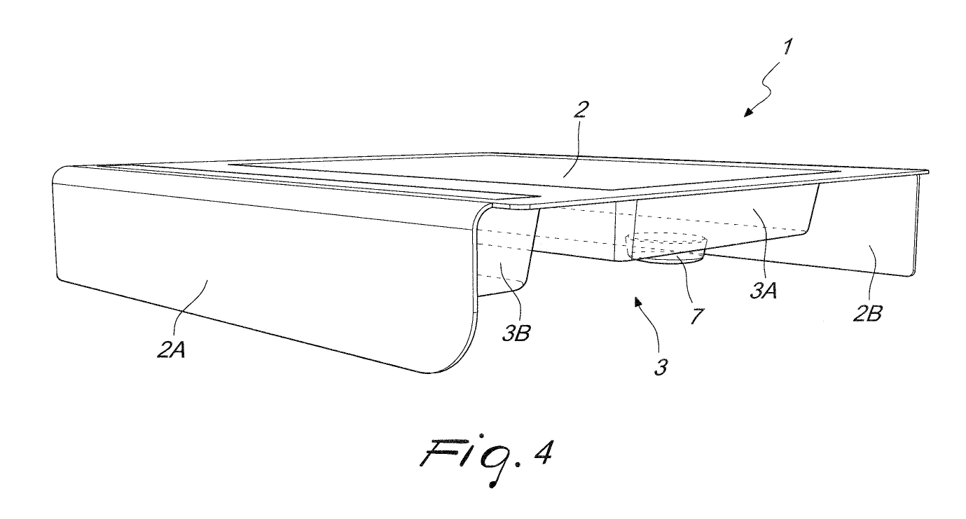

FIG. 4 shows a further perspective view of the second embodiment of the blister packaging according to the invention.

### DESCRIPTION OF PREFERRED EMBODIMENTS OF THE INVENTION

With reference to FIGS. 1 and 2, a first embodiment of the blister packaging, in particular a blister packaging for dental implants or the like, according to the first embodiment of the present invention is described.

The blister packaging generally designated by reference number 1 has a web structure 2 and at least two support wings 2A and 2B which extend perpendicularly to the web structure 2. Although two support wings are shown in the presently preferred embodiment, the provision of additional support wings is conceivable. The web structure is connected to a rear wall (not shown) in a well-known manner for tightly sealing the blister packaging 1.

In the embodiment in FIG. 1, the rear wall has been omitted to allow a view of the plastic film molded part generally designated by the reference number 3, having the compartments described hereafter. It is understood that the rear wall can be manufactured from conventional materials as explained above, such as for instance plastics, cardboard or the like. The web structure 2 is formed integral with the plastic film molded part 3.

The dimensions of the plastic film molded part 3 and of the circumference of the web structure 2, respectively, substantially correspond to the dimension of the circumference of the rear wall; the plastic film molded part 3 is fastened to the rear wall by conventional measures such as e. g. welding, clamping, tacking, adhesive bonding or the like. The height of the support wings 2A and 2B is at least equal to the height of the plastic film molded part 3. Although the support wings 2A and 2B are shown as being continuous parts, they can be discontinuous or provided with openings according to the invention.

In the embodiment in FIGS. 1 and 2, the plastic film molded part 3 is divided into three compartments designated by the reference numbers 3A, 3B and 3C. Obviously, the number of compartments can vary with the products accomodated in them. The shape of the compartments can also be adapted to the products being accomodated in them and can deviate from the substantially quadrangular cuboids shown in the present Figures.

The web structure 2 and the support wings 2A and 2B have sealing profiles 4 which are provided on their surfaces. The sealing profiles 4 are produced when the rear wall is pressed together with the web structure 2 and the support wings 2A and 2B of the blister packaging 1, in the places where the die of the pressing tool is provided with corresponding silicone profiles. The sealing profiles 4 have thermo-adhesive properties for achieving a sealed connection with the rear wall and are arranged in a pattern such as to facilitate a separation of the rear wall from the blister packaging 1; for instance, sealing profile-free areas can be provided on handle portions described hereafter.

On the edges of the web structure 2, handle portions 5 having the form of indentations can be provided for facilitating handling of the blister packaging 1.

As shown in the prolate presentation in FIG. 2, predetermined folding points or predetermined bending points 6 are provided at the respective interfaces of the support wings 2A with the web structure 2, so as to facilitate bending or folding of the support wings 2A and 2B from the prolate position to the position angled at about 90° with respect to the web structure 2. In the angled positions, the transitions from the web structure 2 to the support wings 2A and 2B can be rounded.

For better stacking of the blister packaging 1, respective indentations 8 can be provided in the support wings 2A and 2B.

Moreover, the blister packaging 1 can advantageously be provided with perforations 9 which are dimensioned and positioned such as to facilitate the folding of the support wings 2A and 2B and help to maintain them in the folded position. The folding process can be performed without the delivery of heat. At the same time, the perforations 9 facilitate the possible separation of the support wings 2A and 2B from the blister packaging 1 if a separate storage of the information on the support wings is desired.

The second embodiment of the invention in FIGS. 3 and 4 is identical with the first embodiment in FIGS. 1 and 2, except that there are only two compartments. In addition, protruding button-shaped portions 7 can be provided on the bottom of the compartments which promote better stacking of the blister packaging 1. Therefore, the indentations 8 of the support wings 2A and 2B can be omitted in the second embodiment.

The invention fully achieve the intended objects by providing a novel blister packaging which can be stacked more easily thanks to the support wings and where the individual blister packages can be easily removed from the stack. At the same time, the support wings provide a display surface for information on the received product. The handle portions further facilitate the removal of a blister packaging from the stack. In addition, stacking is supported by the indentations or by the button-shaped portions, respectively.

1-8. (canceled)

9. Blister packaging, in particular blister packaging for dental implants, comprising a web structure to which a rear wall can be fastened sealingly and a plastic film molded part formed integrally with the web structure, wherein the web structure is equipped with at least two support wings that are angled at about 90° with respect to the web structure and extend along opposite sides of the web structure.

10. Blister packaging according to claim 9, wherein the height of the support wings is at least equal to the height of the plastic film molded part.

11. Blister packaging according to claim 9, wherein it is provided, at an edge of the web structure which is free from support wings, with at least one handle portion.

12. Blister packaging according to claim 9, wherein the plastic film molded part has a plurality of compartments.

13. Blister packaging according to claim 9, wherein the transitions from the web structure to the support wings are rounded.

14. Blister packaging according to claim 9, wherein the plastic film molded part has at least one protruding button-shaped portion on its side turned away from the rear wall.

15. Blister packaging according to claim 9, wherein the support wings are equipped with respective indentations.

16. Blister packaging according to claim 9, wherein the support wings can be separated from the blister packaging by means of perforations.
